(19) United States
(12) Patent Application Publication
(10) Pub. No.: US 2010/0055141 A1
(43) Pub. Date: Mar. 4, 2010

(54) PROCESS FOR THE PREPARATION OF COLOURED PARTICULATES

(75) Inventors: Andrea Frances Richardson, Northumberland (GB); John Melvyn Parker, Sheffield (GB); Ian Robert Wheeler, By Kinrose (GB)

Correspondence Address: NIXON & VANDERHYE, PC, 901 NORTH GLEBE ROAD, 11TH FLOOR, ARLINGTON, VA, 22203, US

(73) Assignee: Dunwilco (1198) Limited, Edinburgh (GB)

(21) Appl. No.: 12/595,309

(22) PCT Filed: Apr. 11, 2008
(86) PCT No.: PCT/GB08/01324

§ 371 Date: Oct. 28, 2009

(30) Foreign Application Priority Data
Apr. 11, 2007 (GB) 0707002.2

(51) Int. Cl. B05D 5/06; C04B 14/04; A61K 8/02; C08K 9/00

(52) U.S. Cl. 424/401; 427/215; 106/401; 523/200

(57) ABSTRACT

The present invention provides a process of preparing stained particulates of glass, partially crystalline glass or glass-like amorphous inorganic materials, the process comprising staining particulates of glass, partially crystalline glass or glass-like amorphous inorganic materials that have no dimension greater than 2000 μm.

### BACKGROUND OF THE INVENTION

The present invention relates to the incorporation of colour within a glass or partially crystalline glass particulate, derived from the ancient art of stained glass manufacture.

### Techniques for Colouring Glass

The practice of staining glass, for example to provide stained glass windows in religious buildings, goes back many hundreds of years. It refers to colouration predominantly near to, but within the surface of the glass, by migration of ions and their subsequent reduction within preformed glass. It therefore differs from so-called coloured glass, in which the colour is generated by the addition of colouring agents to a molten glass mixture, resulting in a uniform distribution of the colorant therein. Stained glass is also to be distinguished from coated glass, in which a coloured coating is applied to the surface of the glass, with no appreciable diffusion into the sub-surface region.

Staining of glass has traditionally been carried out with copper or silver compounds, or mixtures thereof, sometimes mixed with other inorganic phases that aid process control, for example clays. They are prepared in slurry or paste form typically by mixing with oils or resins and applied to the surface of the glass to be stained. When subjected to high temperatures, the metal ions of the staining compounds migrate into the glass structure, exchanging with the alkali ions of the glass itself. In this way, they are incorporated into the chemical structure of the glass. At this stage, the ions may not confer strong colouring.

More intense colour is developed in a second stage, in which a chemically reducing environment is provided in which the metal ions are reduced at high temperature to a metal or metal oxide with lower solubility in the glass structure. In some cases, the reducing agent may be a component of the glass itself. In others it may be a reducing atmosphere, also maintained at high temperature.

It is often advantageous to heat the glass that has been coated with a staining formulation in air before exposure to the reducing atmosphere. Any residue from such air exposure may be removed when cool by washing with water or hydrochloric acid prior to the reducing step. The overall thermal regime has a significant influence on the hue and intensity of the colour obtained. An account of the principles of glass staining is provided in “Coloured glasses” by Woldemar A. Weyl, publ. Dawson's, London, 1959, pages 409 to 419 and pages 433 to 435.

More recently, various methods have been described that improve the colour range, colour intensity and/or durability of the ancient art, or simplify the manufacturing process. Thus in U.S. Pat. No. 3,424,567 there is described a three, rather than four stage staining process in which a deep colouration is achieved in glass which contains no reducing agent, for example borosilicate glass.

In a related patent, U.S. Pat. No. 3,420,698, a staining composition comprising cuprous sulphide, silver oxide, lead metaborate and zinc sulphide is described. Firing of this mixture at a sufficiently high temperature on glass produces a deep red staining effect. The colour may be varied by altering the proportions of the components.

In U.S. Pat. No. 3,429,742, a clean, bright red stain was imparted to soda-lime glass by a paste of a copper compound and optionally a minor amount of a silver compound, the process involving three separate periods of treatment at different temperatures.

JP-A-07315884 provides a clear yellow stained glass in which the staining agent is a mixture of copper sulphate, alkaline metal and alkaline earth metal sulphates, silicon dioxide and aluminium sulphate.

Glass Particulates, Partially Crystalline Glass Particulates and their Uses

Particulate glass is produced commercially in two main forms, spheres or beads, which may be solid or hollow, and flakes. Larger diameter solid spheres find use as grinding media, an application for which some hollow types may be unsuited due to their fragility. In smaller diameters, solid spheres, such as the Spheriglass® range manufactured by Potters Industries Inc., are used in plastics where they provide benefits such as reduced shrinkage and warping of mouldings, as well as viscosity reduction of the melt itself.

Hollow spheres, such as the Sphericel® range manufactured by Potters Industries Inc. reduce the viscosity of coatings and are used in barrier films, especially for exterior durability. Such spheres can also enhance light scattering and product opacity in application media. In plastics, hollow glass spheres find use as lightweight fillers. In cosmetics, fine particle size grades provide smoothness.

Glass spheres are also used in light reflecting applications, such as road cones. In such cases, they are typically half silvered to increase light reflection, or applied to an adhesive silvered substrate. Fully silvered glass spheres, such as the Sil-shield range of ECKA Granulate GmbH & Co. KG, find use in electrically conducting applications.

Glass flakes are typically solid and have applications in barrier coatings, especially for exterior applications in aggressive environments, such as bridges over salt water. They extend the life of the coating through improved chemical and wear resistance and the prevention of cracking and peeling. In plastics, they improve physical properties, especially dimensional stability, mechanical properties and abrasion resistance.

The flakes themselves are typically prepared by thinning a continuous molten glass stream, or individual droplets. Thus for example, in EP-A-0289240, there is described a process for the manufacture of flakes of various sizes and materials, including glasses, comprising directing molten material radially from an overflowing cup through parallel spacers, with an air cushion, to control flake thickness. The uniformly thin sheets thus formed are subsequently disintegrated into flakes by mechanical action.

An alternative means of making the flakes is by injecting compressed gas through molten glass, to blow a bubble of thin-walled glass that is pinched by rollers and subsequently disintegrated into flakes. This process is exemplified in, for example, EP-A-1510506.

Colouration of glass particulates expands the applications beyond the functional towards the aesthetic. For aesthetic applications, decorated flakes are generally employed. Thus in the Metashine range of Toyal Aluminium KK, glass flakes of around 1 micron thickness and from 15 to 500 microns diameter are electrolessly plated with silver. The finer particle sizes are incorporated into decorative coatings and plastics. A similar range is offered by Shepherd Color as StarLight®. These products give a glittery or lustrous silver visual effect.

Partially crystalline glass particulates, such as glass ceramics, may also be amenable to the process of the invention. For example, U.S. Pat. No. 2,920,971 describes the production of partially crystalline glasses by limited, but controlled devitrification or crystallisation of glasses. The higher the degree of crystallinity, the more difficult the staining process becomes. Preparation of partially crystalline glass particulates is by introduction of a low concentration of a material, such as an alkali or alkaline earth metal fluoride, that is soluble in the glass melt at high temperatures, but which will crystallise from it at lower temperatures. Small crystals of the compound formed on cooling provide the light diffusion effect, though with only 5 to 10% crystallisation, the overall composition technically remains a glass. The same document goes on to disclose a substantially crystalline, ceramic-type material, in which titanium dioxide as the nucleating species, along with an appropriate thermal regime, are used to induce crystalline phases of 0.1 to 20 microns diameter, substantially throughout the composition. Zirconium dioxide may also be used as a crystal growth nucleating agent.

U.S. Pat. No. 3,528,847 advances the art by disclosing the staining of glass ceramic materials by ion exchanging silver, gold, mercury and other ions with the lithium, sodium and/or potassium ions of the glass ceramic. Again the thermal regime or firing schedule is important in determining the colours obtained.

### DISCLOSURE OF THE INVENTION

According to the invention, there is provided a process of preparing stained particulates of glass, partially crystalline glass or glass-like amorphous inorganic materials, the process comprising staining particulates of glass, partially crystalline glass or glass-like amorphous inorganic materials that have no dimension greater than 2000 μm. The invention also provides stained particulates obtainable by this process.

The invention further provides a process of colouring a material, the process comprising incorporating stained particulates as herein defined into the said material. A plastic, a coating composition and a cosmetic composition each comprising stained particulates as herein defined are further aspects of the invention.

Whilst surface coloured glass particulates are known, the colorant is applied and affixed to the glass surface. Adhesion requirements are therefore high, if the coloured coating is not to be lost in application, for example by the high shear of plastics processing equipment. In contrast, staining of glass particulates avoids this constraint, since the colour lies substantially below the surface. Apart from this high durability characteristic, staining can provide novel visual effects, difficult to attain by surface coating.

The stained particulates of the present invention find their main use for novel aesthetic effects in plastics and surface coatings, especially in applications requiring durability and good light fastness. Stained particulates in optimum particle size ranges further extend the range of novel visual effects in coatings and plastics. In addition however, their functional properties, such as smooth feel in the case of fine particle size, hollow spheres, may be useful in, for example, cosmetics. The silver based and copper based products of the invention may exhibit anti-microbial properties.

### DETAILED DESCRIPTION

As previously mentioned, the present invention provides a process of preparing stained particulates of glass, partially crystalline glass or glass-like amorphous inorganic materials, the process comprising staining glass, partially crystalline glass or glass-like amorphous inorganic particulates that have no dimension greater than 2000 μm.

The term “glass” as used herein encompasses all the main glass types including A-glass, also known as soda or soda-lime glass, chemical or C-glass and borosilicate glass, such as E-glass.

The term “partially crystalline glass” encompasses glass that has up to 10% crystallisation and also encompasses glass that has crystallisation of 10% or more provided that it still exhibits characteristics that are typical of glass, such as a glass transition temperature.

The term “glass-like amorphous inorganic materials” refers to materials that exhibit a disordered structure on an atomic scale but which crystallise at a lower temperature than the glass transition temperature.

There is no criticality to the composition of the glass, partially crystalline glass or glass-like amorphous inorganic materials providing they will withstand the temperatures employed in the staining process without significant structural deformation. Metal ions, preferably monovalent alkali metal ions, are required for the ion exchange process. Thus the main glass types, A-glass, C-glass and borosilicate glass are all suitable.

According to one embodiment, the particulates of the present invention are stained particulates of A-glass, C-glass, borosilicate glass, partially-crystalline glass or glass-like amorphous inorganic materials, more preferably stained particulates of A-glass, C-glass, borosilicate glass or partially-crystalline glass.

Borosilicate glass is more technically challenging to stain than the others. A-glass, which contains calcium oxide and more sodium than potassium, is the most widely used and the least expensive. Thus, in a preferred embodiment, the particulate products of the present invention are stained particulates of A-glass.

In an alternative embodiment, the particulates of the present invention are stained particulates of glass ceramic.

The particulates for use in the present invention may be of any geometry, providing no dimension is greater than 2000 μm (2 mm). Particularly preferred are particulates having no dimension greater than 1500 μm, more preferably those having no dimension greater than 600 μm, even more preferably those having no dimension greater than 100 μm, such as no dimension greater than 70 μm, no dimension greater than 40 μm, no dimension greater than 30 μm, or no dimension greater than 20 μm.

For certain applications, particulates having a narrow particle size distribution are particularly desirable. For example, in coating compositions the brightest effects are generally derived from particulates having a narrow particle size distribution.

Thus, according to one preferred aspect, the particulates have a particle size distribution such that at least 90% by volume of the particulates have a diameter within ±25% of the median diameter of the particulates. In a further preferred aspect, the particulates have a particle size distribution such that at least 90% by volume of the particulates have a diameter within ±20% of the median diameter of the particulates, such as within ±10% of the median diameter of the particulates. Particle size distributions may be measured with a Malvern Master Size Analyser, which is a standard instrument for measuring volume percent particle size distributions.

As regards the shape of the particulates, spheres and flakes are particularly preferred. As previously mentioned, the spheres may be hollow or solid.

The term “flake” refers to a particle having an aspect ratio (that is, the ratio of the largest dimension to the smallest; effectively the diameter to thickness ratio) of at least 3:1. The flakes typically have aspect ratios of between 3:1 and 300:1, preferably between 5:1 and 100:1. In one preferred aspect, the aspect ratio of the flakes is at least 5:1, more preferably at least 15:1.

The process of the invention may be performed with commercially available glass particulates. Alternatively, it may be performed on particulates obtained from the process described and claimed in our co-pending patent application WO 2006/082415 A2, which is discussed further, below.

Staining may be carried out by any process known in the art, requiring the absorption of the staining species into the glass matrix and optionally chemically reducing it in situ, with or without a reducing atmosphere.

In a preferred embodiment the staining comprises the steps of:
(i) applying a staining formulation to the particulates, wherein the staining formulation comprises at least one metal compound; and
(ii) heating the particulates so as to effect migration of metal ions from the staining formulation into the particulates.

The staining formulation comprises at least one metal compound, which may be referred to as the “staining species”. According to one embodiment, the staining formulation comprises at least two metal compounds. Thus, in one preferred aspect, the staining formulation comprises either two silver compounds, two copper compounds or a silver compound and a copper compound, optionally in a carrier.

The staining formulation typically comprises at least one metal compound dispersed or dissolved in a carrier, although the presence of a carrier is not essential. When present, the carrier typically facilitates uniform contact of the staining agent with the glass surface under the staining conditions. The carrier may be any suitable material, for example, those known in the art, such as water, alcohol, organic oil, inorganic oil, or any suitable combination thereof. The active metal compounds for staining may sometimes be mixed with other inorganic phases that aid process control, for example kaolin or calcined clays.

According to one preferred embodiment the staining formulation comprises at least one metal compound in a carrier, preferably in an oil or resin carrier. Examples of the suitable oils are organic oils such as vegetable oil, pine oil and rosin oils.

The staining species may be combined with a carrier in the ratio of 1 part staining species to up to 5 parts carrier. For example, the staining species may be combined with a carrier in a ratio of 1 part staining species to 3 parts carrier or to 2 parts carrier. In an alternative aspect of the invention, the staining species may be combined with the carrier in a ratio of up to 30 parts carrier to 1 part staining species, for instance up to 20 parts carrier to 1 part staining species. In this aspect the ratio is typically greater than 5 parts carrier to 1 part staining species, more typically greater than 10 parts carrier to 1 part staining species.

The staining species, optionally with a carrier, is typically combined with the glass particulates in the ratio of 1 part staining species to up to 100 parts glass particulate by weight, more commonly 1 part staining species to up to 20 parts glass. Typically, a ratio of 1 part staining species to up to 10 parts glass is used, such as 1 part staining species to up to 5 parts glass, or 1 part staining species to 1 part glass. The quantity of staining species used is desirably minimised, subject to acceptable colour development and may be determined by experiment for any combination of staining species and glass composition. A higher level of staining species will generally provide increased colour intensity, but may prove more costly. In some cases, colour saturation may be reached, when further increasing staining concentration will be ineffective.

Amongst suitable staining species are silver and copper compounds, which tend to impart yellow and red shades respectively. In a preferred aspect, the metal compound is a copper compound or a silver compound. The nature of the silver or copper compound does not decisively affect operation of the process. As examples, there may be mentioned inorganic salts and organic salts of silver and copper. Examples of inorganic salts include nitrates, oxides, sulphates, sulphides and chlorides. Examples of organic salts include carbonates and carboxylates such as acetates. Suitable staining species include cuprous chloride and silver nitrate.

Other compounds are optionally added to the staining formulation from which the stained particulates are made, to facilitate the staining reaction or to influence the colour of the resulting stain. For example, inorganic solid phases that assist in process control by acting as diluents, by participating in the reduction process, or by aiding the removal of ions from the glass particulate may be added. Such compounds might include bismuth, tin, vanadium, zirconium, lithium or iron, or silicate minerals, such as clays or other aluminosilicates.

Other compounds may also be present in the glass particulates to enhance migration of the metal ions, to facilitate the staining reaction, or to further modify the stain-derived colour. Thus, for example, lead oxide and barium oxide may provide more favourable glass staining conditions when part of the glass composition than the more commonly employed calcium oxide formulations. Minor quantities of compounds of aluminium, antimony, arsenic, boron, carbon (graphite), cerium, chromium, cobalt, copper, gold, iron, magnesium, manganese, molybdenum, nickel, platinum, selenium, silver, titanium, vanadium, zinc, zirconium and rare earths present in the glass batch composition may affect colour development in the final glass, without impairing other features of the process.

The thermal treatment regime, both temperature and time, has a significant influence on the nature of the colour effect obtained. Whilst the optimum thermal regime may be found by experiment for a given formulation, the temperature should not exceed the nominal softening point of the glass. Optimum temperatures normally lie between 100° C. and 800° C., often 400° C. to 650° C. Thus, in one preferred embodiment, the particulates are heated at a temperature of 100° C. to 800° C., preferably 400° C. to 650° C., preferably within glass transition temperature, Tg, +/−200° C., more preferably +/−100° C. Thus, a temperature above or below Tg may be used, though temperatures below Tg are preferred. In general, temperatures of 250° C. and above are preferred.

Treatment times may be extended at lower temperatures. A few minutes to 20 hours or more may be required for the desired colour to be generated. A satisfactory first stage result can often be achieved from a thermal regime of around 550 to 700° C. for copper and 450 to 600° C. for silver, up to five hours, typically in between two and five hours. The reduction step typically operates at similar temperatures, but for a shorter time, often under one hour. Higher temperatures are generally required to reduce divalent ions.

According to one preferred aspect, the process further comprises the step of reducing the metal ions. The metal ions may be reduced to any lower oxidation state, i.e. the metal ions may be either partially or fully reduced. For example, a metal ion in the +2 oxidation state (e.g. Cu²⁺) may be reduced to the +1 oxidation state (e.g. Cu⁺) or the zero oxidation state (e.g. elemental copper).

The metal ions are typically reduced by heating the particulates in the presence of a reducing agent. The reducing agent may be either in or on the particulates. For example, silver ions can be easily reduced by reducing agents already present in the glass. Arsenic, antimony and iron ions all facilitate this reaction. Alternatively the reducing agent may be present in the staining formulation or may be applied separately to the glass particulates. A further alternative is for the reducing agent to be present in a reducing atmosphere. Hydrogen is the most economical means of providing a reducing atmosphere, but carbon monoxide is also used. In both cases, an inert carrier gas is advantageously employed, at up to 99% by volume of the total, typically 95 to 97%. Sulphur dioxide provides a milder reducing atmosphere. Whilst such a reducing atmosphere is normally required for copper staining, silver ions can be easily reduced by reducing agents already present in the glass.

According to one preferred aspect, the process further comprises the step of washing the particulates. This may be done to remove any undesirable residues from the staining formulation. The particulates are typically washed in water, organic or inorganic acids, or bases. Examples of suitable acids include hydrogen fluoride, hydrogen chloride and nitric acid. Examples of suitable bases include alkaline or alkaline earth hydroxides such as sodium hydroxide. Preferably the particulates are washed in water or dilute acid. The washing stage may be carried out before or after a reduction step, or both. A brightening of colour and/or a shade change may be observed.

As previously mentioned, the process of the invention may be performed with commercially available glass particulates. Alternatively, it may be performed on particulates obtained from the process described and claimed in our co-pending patent application WO 2006/082415 A2. There is described therein a process for preparing non-metal particulates that comprises the steps of:
(i) printing a liquid precursor of a non-metal particulate onto a collecting substrate,
(ii) subjecting the non-metal precursor to a solidification treatment to provide non-metal particulates and
(iii) recovering the non-metal particulates.

The liquid precursor may be printed by any suitable printing method. Suitable printing methods allow the printing of discrete shapes of sufficiently small quantities of the precursor that the resultant non-metal particulates have no dimension greater than 2000 μm. Smooth-surfaced, flake-shaped particulates are particularly preferred. Where a silica-forming material, such as a sol-gel, is employed as the printable liquid precursor, the derived glass particulates, if of a composition that includes the metal ions of the invention, are amenable to the process of the present invention. In a further embodiment, the non-metal particulates may be stained before recovery from the collecting substrate.

In a preferred embodiment, the liquid precursor is a sol-gel, such as tetraethyl orthosilicate and the solidification treatment comprises heating at a temperature up to 1100 to 1200° C., that is sufficient to fuse the sol-gel into vitreous silica or a suitable derivative.

### Other Aspects

The present invention provides stained particulates obtainable by any of the processes of the invention.

In one broad aspect, the present invention provides stained particulates of glass, partially crystalline glass or glass-like amorphous inorganic materials having no dimension greater than 2000 μm. Such particulates are preferably prepared by a process as herein defined. When prepared by this process the stained particulates each have no dimension greater than 2000 μm and typically each comprise a coloured stain which is present throughout the surface and sub-surface regions of the particulate, the concentration of the coloured stain decreasing with increasing distance from the surface of the particulate.

The present invention also provides a process of colouring a material, the process comprising incorporating stained particulates as herein defined into the material. The material may advantageously be a plastic in particular a thermoplastic, a coating composition such as a paint or ink, or a cosmetic composition. Thus the present invention provides plastics, coating compositions and cosmetic compositions comprising stained particulates as herein defined.

In one aspect of the present invention, the particulates which undergo the staining process of the present invention are uncoloured. However, in an alternative aspect they are coloured, which can lead to interesting visual effects. When they are coloured, the coloured particulates to be stained are obtainable, for instance, by a previous staining step or by incorporation of the colouring species into the bulk phase precursor material from which the particulates are formed. In the case where the colour is present from a previous staining step, said previous staining step is typically a staining process of the present invention as described herein. In the case where the colour is present due to the colouring species having been incorporated into the bulk phase precursor material from which the particulates are formed, the coloured particulates to be stained are typically obtainable using the same methods as are described below for the incorporation of metal ions into a bulk phase precursor material, as part of the “final aspect” of the present invention.

Whereas the present invention in earlier aspects is concerned with forming coloured particulates by a staining process by which colour is developed, which may (but does not have to) involve a reduction step, a final aspect of the invention is concerned with forming coloured particulates by use of a reduction step to develop the colour. According to this final aspect, the present invention provides a process of preparing particulates of glass, partially crystalline glass or glass-like amorphous inorganic materials, the process comprising reducing metal ions located in particulates of glass, partially crystalline glass or glass-like amorphous inorganic materials, which particulates have no dimension greater than 2000 μm. Prior to this reduction step, the metal ions in the particulates may or may not give rise to colour, though typically they do not.

Whereas in the earlier aspects metal ions responsible for the colouration are added to the particulates by staining pre-formed particulates and development of the colouration, in the final aspect the metal ions are generally present in the bulk phase precursor material from which the particulates are formed. Accordingly, the final aspect will generally involve the step of converting a bulk phase precursor material containing metal ions into particulates of glass, partially crystalline glass or glass-like amorphous inorganic materials that have no dimension greater than 2000 μm.

Further, the final aspect may involve a step of admixing the metal ions responsible for the colouration into a bulk material in order to form the bulk phase precursor material. Accordingly, the final aspect may also comprise a preliminary step of mixing the metal ions or a metal compound which provides the metal ions, with at least one other component of the bulk phase precursor material.

There is no particular limitation to the nature of the bulk phase precursor material, except that it should be able to accommodate metal ions and is not composed of individual solid particulates. For instance, if glass particulates are desired, the bulk phase precursor material is preferably molten glass. In this case metal ions can be incorporated into the glass when molten, e.g. by simple mixing, or mixed with other ingredients for forming the glass prior to melting.

In one embodiment, when particulates in the form of glass flakes are desired, they can be prepared by thinning a continuous stream of a molten glass mixture containing the metal ions. For instance, they can be prepared by injecting compressed gas through the molten glass mixture containing the metal ions, blowing a bubble of thin-walled glass that is pinched by rollers, with subsequent disintegration into flakes.

In another embodiment of this final aspect, glass particulates comprising metal ions can be prepared from an appropriate glass precursor by a printing process as described in WO2006/082415. In this embodiment the precursor may be a sol gel or a low melt temperature glass containing the metal ions.

In this final aspect (i) the metal ions are typically derived from a metal compound as described herein for the metal compound (“staining species”) as used in other aspects of the present invention; (ii) the reduction step and preferred aspects thereof are the same as those described above; (iii) the bulk phase precursor material and the particulates obtainable therefrom can optionally include other compounds as described above (although compounds which serve only to facilitate the staining reaction are not needed); and/or (iv) the preferred sizes of the particulates are the same as those described above.

The particulates of glass obtainable by the process of the final aspect of the present invention are typically coloured particulates, and as with earlier aspects of the invention the reduction step typically causes a change or an enhancement of colour. In all aspects of the present invention involving the reduction of metal ions within the particulates, the reduction step can lead to the presence of the product(s) of reduction of the metal ions (i) within the particulate at a concentration which changes (e.g. increases or decreases) with increasing distance from the surface, and (ii) on the surface of the particulate. The “product(s) of reduction of the metal ions” can be elemental metal and/or metal compound(s) and the product(s) on the surface may be the same as or different to those within the particulate. This is true also of earlier aspects of the invention.

Thus, the present invention provides particulates of glass, partially crystalline glass or glass-like amorphous inorganic materials, which particulates have no dimension greater than 2000 μm, and which particulates have metal and or metal compounds (i) within the particulate at a concentration which changes (e.g. increases or decreases) with increasing distance from the surface, and (ii) on the surface of the particulate, and processes for making such particulates.

The invention is further illustrated by the following Examples in which all parts and percentages are by weight, unless otherwise stated.

### EXAMPLES

### Example 1

Solid glass spheres of approx. 5 to 7 μm median diameter (Spheriglass® 5000 of Potters Industries Inc.) (20 g) and cuprous chloride (15 g) were added to a crucible and mixed. The crucible was added to a furnace and heated. The temperature was ramped to 650° C. over approx. 4 hours, then maintained at this temperature for a further 3 hours. The sample was then removed, cooled and washed in 5% dilute hydrochloric acid solution, then vacuum filtered. The sample was thereafter heated, by similarly ramping to 650° C., and held 30 minutes in a reducing atmosphere of 5% hydrogen in nitrogen, then recovered in the same way, finally drying to a powder. This produced a red glass sphere.

### Example 2

The method of Example 1 was repeated with substitution of solid glass spheres (20 g) by hollow glass spheres (Sphericel® 60P18 of Potters Industries Inc.) of approx. 18 μm diameter (5 g) and 5 g of cuprous chloride was used in place of 15 g. This also produced a red glass sphere, of a slightly paler colour than that of Example 1. An additional washing step was carried out, using 10% dilute hydrochloric acid solution. This was found to be advantageous for the surface appearance and colour.

### Example 3

Glass flakes (GF300M of Glassflake Ltd.) (4 g) and cuprous chloride (4 g) were added to a crucible and mixed. The method of Example 1 produced a bright metallic red glass flake, with a smooth, reflective surface. A range of red colours can be produced using this method by altering the process conditions. An additional washing step can optionally be employed, using dilute hydrochloric acid solution, to give improved surface appearance and colour.

### Example 4

Glass flakes (GF300M of Glassflake Ltd.) (3 g) and cuprous chloride (3 g) were added to a crucible and mixed. The crucible was added to a furnace and heated. The temperature was ramped to 600° C. over approx. 4 hours, then maintained at this temperature for a further 3 hours. The sample was removed, cooled and washed in 5% dilute hydrochloric acid solution, and then vacuum filtered. In the absence of the reducing step, this produced a green glass flake. The shade of green obtained, as well as the ability to produce a range of related colours, can be varied by altering the process conditions.

### Example 5

Silver nitrate (0.4 g) was dissolved in de-ionised water (7.5 g). Glass flakes (3 g) (GF300M of Glassflake Ltd.) were then added and the crucible inserted in a furnace. The temperature was raised to 80 to 100° C. and held for approx. 30 minutes. Thereafter, the temperature was ramped to 550° C. over 3 hours and maintained at that temperature for 40 minutes. The flakes contained in the crucible were stirred using a glass rod at intervals during the ramping process. The sample was thereafter removed, cooled and washed in de-ionised water, followed by vacuum filtration and drying. A pale gold coloured flake was obtained.

### Example 6

The procedure of Example 5 was repeated but the sample was subsequently ramped in the furnace to 500° C. and held for 30 minutes in a reducing atmosphere containing 5% hydrogen. This produced a rich gold coloured glass flake. As in earlier examples, adjusting the process conditions can alter the colour produced using this method.

### Example 7

Crystal polystyrene (200 g), the flake of Example 3 (0.2 g) and DOA (Di-Octyl-Adipate) (0.3 g) were mixed and injection moulded to produce a plastic sample using typical moulding conditions for polystyrene. This produced a clear plastic matrix with discrete particles of red flake incorporated therein. On adding the flake to a white coloured polypropylene in a similar manner, a white matrix with discrete particles of coloured flake was produced. Thus no bleeding of colour was apparent in either case.

### Example 8

A solvent-borne coating was produced using flake manufactured by the procedure of Example 3. The coating was then spray applied to the surface of an unprimed steel panel. After stoving, this produced a durable coating with good hiding power and a pronounced red metallic colour. A clear topcoat may optionally be applied. In such an instance, the coating exhibits a change of hue and reflection with changing viewing angle. The products of Example 3 also produced a novel visual effect when applied to a black base coated panel, then clear topcoated.

### Example 9

A powder coating was produced using a red flake, a combination of GF300M and GF003 glass flake (a glass flake of finer median particle size, also from Glassflake Ltd.), which had been produced using the procedure of Example 3. The flake had been sieved so that only a size distribution lower than 45 μm was being used. A clear powder coating material (135.5 g) was mixed with the red flake (14.5 g) and the powder coating was applied to steel panels by electrostatic spraying. This produced a metallic red powder coating, with an attractive, subtle level of sparkle.

### Example 10

The procedure of Example 9 was repeated to produce a powder coating which contained a red glass flake and aluminium flake material, incorporated into a clear powder coating resin. A clear powder coating resin (138.3 g) was mixed with a combination of GF300M and GF003 glass flake (5.8 g), which had been produced using the procedure of Example 3. The flake had been sieved so that only a size distribution between 45 and 100 μm was being used. An aluminium flake (SBC PC 3101X of Silberline Ltd.) (5.84 g) was then added to the mixture and the combination of materials was sprayed. This produced an attractive and unusual coating containing discrete red and silver colours, but also exhibiting metallic travel.

### Example 11

Red hollow spheres (18 microns diameter Sphericel 60P18 of Potters Industries Inc.) were produced using the procedure of Example 1. These flakes were then incorporated into a lip balm material. Vaseline (Vaseline pure petroleum jelly, a brand of Unilever) (3.5 g) was heated for approximately one minute and then mixed with the red spheres (0.2 g). This produced a deep red coloured, smooth lip balm.

### Example 12

Red hollow spheres (18 microns diameter Sphericel 60P18 of Potters Industries Inc.) were produced using the procedure of Example 1. These particulates were formulated as an ink by mixing the red spheres (1 g) with isopropanol (2.5 g) and Glascol LS2 (6 g), (a water-carried acrylic resin manufactured by Ciba Speciality Chemicals). A drawdown was then produced using a No. 4 wire-wound bar. A rich red effect was obtained.

### Example 13

A glass batch having the composition set out in Table 1 or 2 is melted and compressed gas is injected through the molten glass mix, to blow bubbles of thin-walled glass that are pinched by rollers and subsequently converted into glass particulates containing silver ions. The particulates are refined to remove those with a diameter greater than 2000 μm, and then subjected to a temperature of 550° C., for 30 minutes, in a reducing atmosphere of 5% hydrogen in nitrogen. The particulates are then recovered by removal, cooling, washing in 5% dilute hydrochloric acid solution, and vacuum filtering. Finally the particulates are dried to a powder.

TABLE 1

| Component | Parts |
|---|---|
| Sand | 1000 |
| Sodium carbonate | 300 |
| Calcium carbonate | 300 |
| Sodium nitrate | 30 |
| Sodium chloride | 25 |
| Sodium sulphate | 5 |
| Aluminium oxide | 21 |
| Boric oxide | 30 |
| Arsenic oxide | 15 |
| Silver nitrate | 11.25 |
| Manganese oxide | 3.75 |

TABLE 2

| Component | Parts |
|---|---|
| Sand | 950 |
| Sodium carbonate | 210 |
| Potassium carbonate | 140 |
| Feldspar | 160 |
| Calcium carbonate | 160 |
| Zinc oxide | 150 |
| Boric oxide | 60 |
| Silver nitrate | 12 |
| Sodium urinate | 4 |
| Arsenic oxide | 4 |
| Manganese oxide | 4 |

### Example 14

Particulates are prepared using the same method and components as in EXAMPLE 13, except that instead of having compressed gas injected through it, the molten glass mix is directed radially from an overflowing cup through parallel spacers, with an air cushion, to control flake thickness. The resulting uniformly thin sheets are subsequently disintegrated into glass particulates containing silver ions. The particulates are then reduced as in EXAMPLE 13.

### Example 15

Particulates are prepared using the same method and components as in EXAMPLE 13 or 14, except the reduction temperature is 450° C. instead of 550° C.

1. A process of preparing stained particulates of glass, partially crystalline glass or glass-like amorphous inorganic materials, the process comprising staining particulates of glass, partially crystalline glass or glass-like amorphous inorganic materials that have no dimension greater than 2000 μm.

2. A process according to claim 1 wherein the particulates have no dimension greater than 600 μm.

3. A process according to claim 1 wherein the particulates have no dimension greater than 30 μm.

4. A process according to claim 1 wherein the particulates have a particle size distribution such that at least 90% by volume of the particulates have a particle diameter within ±25% of the median particle diameter.

5. A process according to claim 1 wherein the particulates are in the form of spheres or flakes.

6. A process according to claim 5, wherein the particulates are hollow spheres.

7. A process according to claim 5, wherein the particulates are solid spheres.

8. A process according to claim 5, wherein the particulates are flakes.

9. A process according to claim 8, wherein the flakes have an aspect ratio of from 3:1 to 300:1.

10. A process according to claim 8, wherein the flakes have an aspect ratio of from 5:1 to 100:1.

11. A process according to claim 1 wherein the particulates of glass, partially crystalline glass or glass-like amorphous inorganic materials contain alkali metal ions.

12. A process according to claim 11 wherein the particulates are A-glass.

13. A process according to claim 1 wherein the particulates are of glass ceramic.

14. A process according to claim 1 wherein the staining comprises the steps of:
(i) applying a staining formulation to the particulates, wherein the staining formulation comprises at least one metal compound; and
(ii) heating the particulates so as to effect migration of metal ions from the staining formulation into the particulates.

15. A process according to claim 14 wherein the staining formulation comprises at least one metal compound in an oil or resin carrier.

16. A process according to claim 14 wherein the metal compound is a copper compound or a silver compound.

17. A process according to claim 14 wherein the staining formulation further comprises bismuth, tin, vanadium, zirconium, lithium or iron, or silicate minerals.

18. A process according to claim 14 wherein, in step (ii), the particulates are heated at a temperature of 100° C. to 800° C., preferably 400° C. to 650° C.

19. A process according to claim 14 which further comprises the step of:
(iii) reducing the metal ions.

20. A process according to claim 19 wherein the metal ions are reduced by heating the particulates in the presence of a reducing agent, wherein the reducing agent is either in or on the particulates or is present in a reducing atmosphere.

21. A process according to claim 14 which further comprises the step of washing the particulates after the heating step (ii) either prior to the reducing step (iii), after the reducing step (iii), or both.

22. A process according to claim 21 wherein the particulates are washed in either water or dilute acid.

23. A process of preparing particulates of glass, partially crystalline glass or glass-like amorphous inorganic materials, the process comprising reducing metal ions in particulates of glass, partially crystalline glass or glass-like amorphous inorganic materials, which particulates have no dimension greater than 2000 μm.

24. A process according to claim 23, which process comprises the steps of
(i) incorporating metal ions into a bulk phase precursor material of particulates of glass, partially crystalline glass or glass-like amorphous inorganic materials,
(ii) converting said bulk phase precursor material into particulates of glass, partially crystalline glass or glass-like amorphous inorganic materials, which particulates have no dimension greater than 2000 μm, and
(iii) reducing metal ions in the particulates.

25. A process of preparing stained glass particulates, the process comprising the steps of:
(i) printing a liquid precursor of a glass particulate onto or into a collecting substrate;
(ii) subjecting the precursor to a solidification treatment to provide glass particulates that have no dimension greater than 2000 μm;
(iii) recovering the glass particulates; and
(iv) staining the glass particulates.

26. A process according to claim 25 wherein the particulates having no dimension greater than 600 μm and the staining process comprises the steps of:
(i) applying a staining formulation to the particulates, wherein the staining formulation comprises at least one metal compound; and
(ii) heating the particulate so as to effect migration of metal ions from the staining formulation into particulates.

27. Particulates obtained or obtainable by the process of claim 1.

28. Stained particulates of glass, partially crystalline glass or glass-like amorphous inorganic materials having no dimension greater than 2000 μm.

29. Particulates of glass, partially crystalline glass or glass-like amorphous inorganic materials, which particulates have no dimension greater than 2000 μm, and which particulates have metal and or metal compounds (i) on the particulate surface and (ii) within the particulate at a concentration which changes with increasing distance from the surface.

30. A process of colouring a material, the process comprising incorporating particulates as defined in claim 28 into the material.

31. A process of colouring a material, the process comprising:
(i) preparing particulates by a process as defined in claim 1; and
(ii) incorporating the particulates in the material.

32. A process according to claim 30 wherein the material is a plastic.

33. A process according to claim 30 wherein the material is a coating composition.

34. A process according to claim 30 wherein the material is a cosmetic composition.

35. A plastic comprising particulates as defined in claim 28.

36. A coating composition comprising particulates as defined in claim 28.

37. A cosmetic composition comprising particulates as defined in claim 28.
